(19) United States
(12) Patent Application Publication
(10) Pub. No.: US 2009/0090152 A1
(43) Pub. Date: Apr. 9, 2009

(54) SYSTEMS AND METHODS FOR PROVIDING CONTROL AND DISPOSAL OF WASTE MATERIALS

(75) Inventors: Charles W. Kallmann, Provo, UT (US); Edward T. Woodruff, Brookville, FL (US); Kenneth S. Marsh, Seneca, SC (US)

Correspondence Address: KIRTON AND MCCONKIE, 60 EAST SOUTH TEMPLE,, SUITE 1800, SALT LAKE CITY, UT, 84111, US

(73) Assignee: Global Sanitation Solutions, Inc.

(21) Appl. No.: 12/062,475

(22) Filed: Apr. 3, 2008

Related U.S. Application Data
(60) Provisional application No. 60/922,068, filed on Apr. 4, 2007.

(51) Int. Cl. G05F 3/00; A61L 2/16; B09B 3/00; C12M 1/33; C12S 13/00; G05D 16/00

(52) U.S. Cl. 71/12; 422/28; 422/184.1; 422/113; 435/262.5; 435/283.1

(57) ABSTRACT

Systems and methods for managing and disinfecting excrement material. A device includes a biopolymer decomposition container. The method includes treating an excrement sample with a bioactive agent, providing oxygen to the bioactive agent, and disinfecting microorganisms present within the system. The system and method further provides a byproduct that may be used to enrich soil.

### RELATED APPLICATIONS

This application claims priority to U.S. Provisional Patent Application Ser. No. 60/922,068 filed Apr. 4, 2007, entitled SYSTEMS AND METHODS FOR PROVIDING CONTROL AND DISPOSAL OF HUMAN WASTE MATERIALS, which is incorporated herein by reference.

### BACKGROUND OF THE INVENTION

1. Field of the Invention

The present disclosure relates to waste management and waste disinfection. More particularly, the present disclosure pertains to systems and methods for disinfecting excrement, including systems and methods for recycling excrement into a usable product.

2. Background and Related Art

Current technologies are available to deodorize human excrement. Such technologies are used in the camper, aircraft, bus, and portable toilet industries. While such technologies currently exist to deodorize human excrement, there is a need to control the spread of harmful pathogens contained in both human and non-human excrement.

Accordingly, it would be an improvement in the art to augment, or even replace, current techniques with other techniques.

### SUMMARY OF THE INVENTION

The present disclosure relates to waste management and waste disinfection. More particularly, the present disclosure pertains to systems and methods for disinfecting excrement, including systems and methods for recycling excrement into a usable product.

At least some implementations of the present invention take place in association with human and/or animal excrement. More specifically, at least some implementations of the present invention take place in association with a system and method for disposing, decomposing, and disinfecting an excrement sample. In some implementations, the system includes a container for collecting an excrement sample. In further implementations, the container generally includes a biopolymer material that is configured or arranged to receive an excrement sample. For example, in some implementations, the container is a bag. Additionally, in some implementations, the container is flat sheet.

In some implementations, the container is positioned adjacent to a user, such that an excrement sample from the user is directly received by the container. Alternatively, in some implementations, an excrement sample is transferred to the container from another location.

In some implementations, the container further contains a bioactive agent for decomposing the excrement sample. The bioactive agent generally includes a microorganism, or mixed culture of microorganisms capable of digesting and decomposing the various components of the excrement sample. In some implementations, the bioactive agent further includes a non-microorganistic entity, such as an enzyme. In some implementations the bioactive agent is provided in a powdered form, while in other implementations the bioactive agent is provided in a liquid form. The bioactive agent may also be lyophilized and vacuum sealed to preserve the bioactivity of the agent. In some implementations, the bioactive agent is applied to the container prior to collecting the excrement sample. In other implementations, the bioactive agent is applied directly to the excrement sample following collection of the excrement in the container.

The container may further include a chemical oxidant component and/or chemical agents to generate oxidizing components. In some implementations, a chemical oxidant component is included in the system to provide oxygen to the aerobic bioactive agent. Additionally, in some implementations, a byproduct of the chemical oxidant is used to disinfect the bioactive agent and any other microorganism within the system.

The container may further include multiple compartments containing various components of the system. For example, in some implementations, a first compartment is provided to contain the excrement sample and the bioactive agent. In some implementations, a second compartment is provided to contain the chemical oxidant and/or oxidant generator. The compartments of the container may further include means for allowing the transfer of oxygen from one compartment to another compartment. In some implementations, a third compartment is provided to contain a second bioactive agent of the system.

The compartments of the container may further include biopolymer materials having various biodegradation properties. For example, in some implementations, a first material having a first biodegradation rate is positioned between the first compartment and the second compartment. Furthermore, a second material having a second biodegradation rate is provided to contain the first and second compartments, where the second biodegradation rate is slower that the first biodegradation rate. As such, upon biodegradation of the first material, the contents of the first compartment and the second compartment are combined and contained within the second material. In some implementations, a third material having a third biodegradation rate is positioned between a second bioactive agent and the other components of the system. Differential degradation rates may be obtained through alternate structures of biopolymers as well as by lamination or coextrusion of biopolymers.

In some implementations of the present method, a first step is to collect an excrement sample. In some implementations, other steps include treating the excrement with a bioactive agent, providing oxygen to the bioactive agent, disinfecting the bioactive agent and pathogens of the system with a disinfecting agent, and disposing the byproduct of the system. In some implementations, another step includes treating the unmetabolized excrement sample with a second bioactive agent.

In some implementations of the present method for manufacturing the container, a first step is to select a raw material or materials for the container. In some implementations, other steps of manufacture include extruding, coextruding and/or laminating the raw materials, providing a label to the extruded materials, forming the container from the extruded materials, loading the various compartments of the container, and sealing the container.

While the methods and processes of the present invention have proven to be particularly useful in the area of waste management and treatment, those skilled in the art can appreciate that the methods and processes can be used in a variety of different applications for managing and disinfecting excrement.

These and other features and advantages of the present invention will be set forth or will become more apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a representative flow diagram attending to the collecting, decomposing, disinfecting, and disposing of an excrement sample;

FIG. 2A illustrates a plan view of a representative embodiment of a decomposition container;

FIG. 2B illustrates a cross-sectional end view of a representative embodiment of a decomposition container;

FIG. 2C illustrates a cross-sectional end view of a representative embodiment of a decomposition container;

FIG. 3A illustrates a partially cross-sectioned perspective view of a representative embodiment of a decomposition container;

FIG. 3B illustrates a partially cross-sectioned perspective view of a representative embodiment of a decomposition container having separate packets; and

FIG. 4 illustrates a representative flow diagram detailing the steps for manufacturing the decomposition container.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed, but is merely representative of presently preferred embodiments of the invention.

As provided herein, the present disclosure relates to waste management and waste disinfection. More particularly, the present disclosure pertains to systems and methods for disinfecting excrement, including systems and methods for recycling excrement into a usable product.

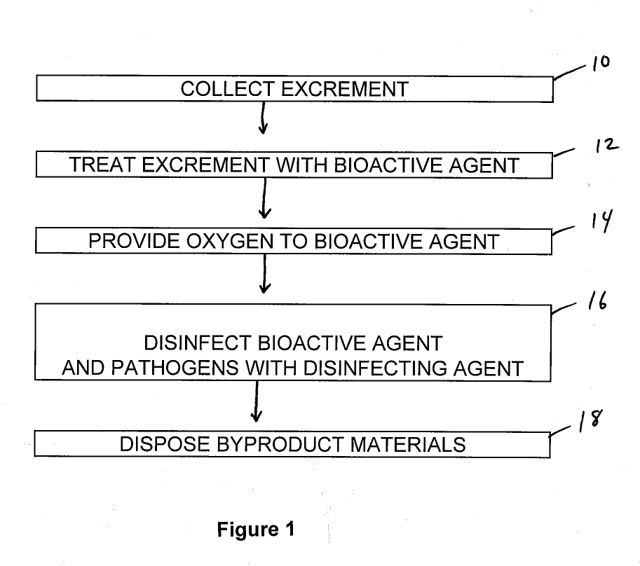

Referring now to FIG. 1, a representative method for disinfecting an excrement sample is provided. An excrement sample generally includes waste products of metabolism and other non-useful materials. Specifically, excretory products include urine and feces, but may also include blood, vomit, mucus, and other forms of bodily discharge. Urine and feces is generally composed of unmetabolized food, including proteins, carbohydrates, fats, and cellulose, as well as water, bacteria, salts, bile, indole, and skatole. Additional components may include blood, mucus, and any variety of pathogens, including viruses, parasites, harmful bacteria, and fungi. All living creatures produce excrement in one form or another. The present method is directed toward disinfecting human excrement samples, but may also be used for disinfecting excrement samples of other species. For example, the present method may effectively be used to disinfect an excrement sample of a dog, a cat, a horse, a cow, a pig, a chicken, or any other excrement producing species.

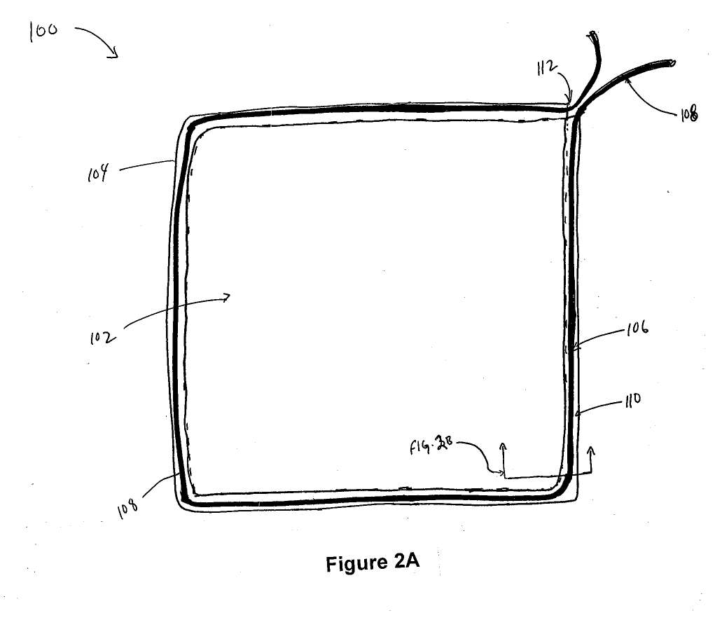

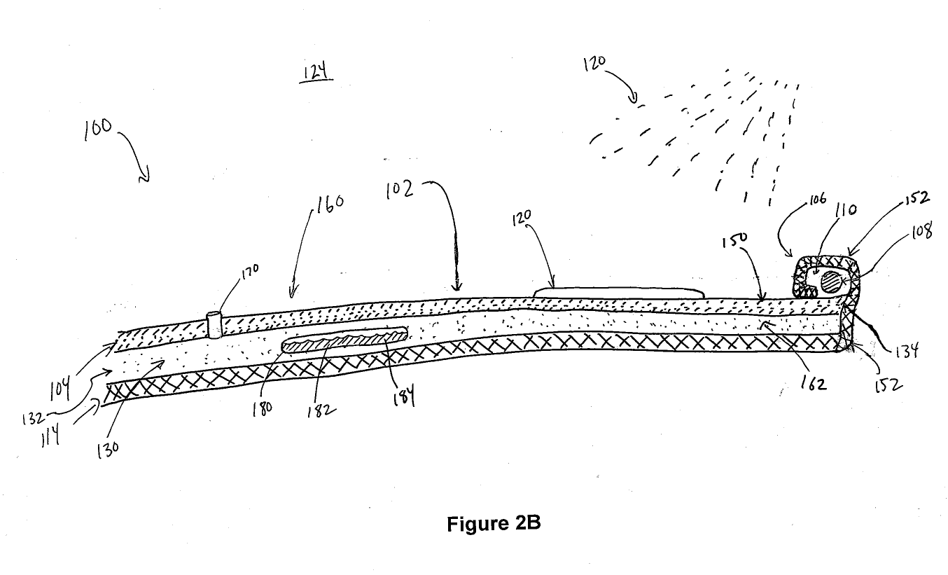

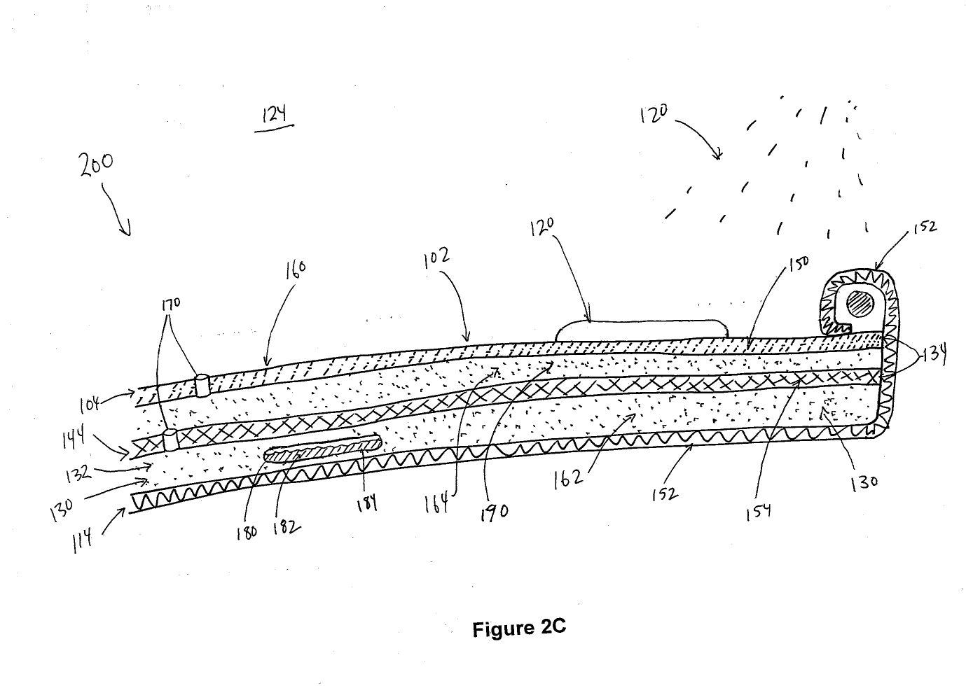

The first step 10 of the method is to collect the excrement sample. The excrement sample is collected in a decomposition container. The decomposition container may include any device capable of containing the excrement sample and the other components of the disinfection system, as described in detail below. In one embodiment, the decomposition container 100 comprises multiple layers of sheets as illustrated in FIGS. 2A-2C.

Referring now to FIG. 2A, a representative perspective top view of the decomposition container 100 is illustrated. The decomposition container 100 is generally planar having a generally rectangle shape. However, other planar shapes may effectively be used. For example, the decomposition container 100 may include a round or rectangular shape. An upper surface 102 of the decomposition container 100 is positioned to receive the excrement sample. The upper surface 102 comprises the upward facing surface of the top layer 104 of the decomposition container 100. The material and characteristics of the top layer 104 will be discussed in detail below.

The decomposition container 100 further comprises a means 106 for enclosing the excrement sample within the container 100. The means 106 may include any method sufficient to retain the excrement. For example, the means 106 may include a drawstring 108, as illustrated. The top layer 104 may be modified to include a channel 110 within which the drawstring 108 may be located. The channel 110 may be provided by folding a portion of the top layer 104 back onto itself. A section of the folded portion of the top layer 104 may then be attached to the upper surface 102 of the top layer 104 by an appropriate method.

For example, the folded portion of the top layer 104 may be secured with an adhesive, or may be secured by melting together a portion of the two adjacent surfaces. Where a drawstring 108 is selected as the means 106, an opening 112 may be provided in the channel 110 to permit the user the access and actuate the drawstring 108 for securing the excrement. Alternatively, two or more openings may be provided to facilitate closing the container 100. The means 106 may also include an adhesive, a mating channel closure, and any other appropriate method for securing the excrement sample. Alternatively, the channel 110 may be provided by folding and attaching a portion of a separate layer onto the top layer 104 of the decomposition container 100, as shown in FIG. 2B.

The excrement may be collected in the decomposition container 100 either directly from the donor, or by transferring the excrement from a primary location to the decomposition container 100. To facilitate the collection of the excrement, the decomposition container 100 may be positioned proximal to the donor during elimination of the excrement. For example, where the excrement is eliminated into a secondary container, such as the bowl of a toilet, the decomposition container 100 may be positioned within the bowl of the toilet directly beneath the seating surface of the toilet. As such, the excrement may be collected directly into the decomposition container 100. Alternatively, where the excrement is initially deposited outside the decomposition container 100, the excrement may be collected and deposited into the decomposition container 100 by any appropriate means.

Referring now to FIG. 2B, a representative cross-sectional end view of the decomposition container 100 is illustrated. The decomposition container 100 comprises a top layer 104 and a bottom layer 114. The top layer 104 comprises a first material 150, and the bottom layer 114 comprises a second material 152. In one embodiment, the first and second materials 150 and 152 include a polymer, or bioplastic capable of performing according to the embodiments of the present invention. In one embodiment, the polymer material of the first and second materials 150 and 152 includes a polyhydroxyalkonate (PHA).

PHAs are linear, biodegradable polyesters of various hydroxyalkonates. PHAs are most commonly synthesized and intracellularly accumulated by numerous microorganisms as energy reserve material. The mechanical properties of PHAs are highly dependent on the constituting monomer units and molecular weight. More than 150 different monomer units have been identified as the constituents of PHAs. These monomers can be combined to produce materials with extremely different properties.

PHAs are biopolymers chains comprising variations of the monomer unit as shown in diagram 1. The R group of the monomer may be substituted by a wide range of organic molecules. For example, R can be substituted with hydrogen or hydrocarbon chains of up to around C13 in length, and n can range from 1 to 3, or more. Therefore, when R is a methyl group and n=1, the polymer is poly-(3-hydroxybutyric acid) (PHB). Alternatively, when R is a methyl group and n=0, the polymer is polylactic acid (PLA), and when R is a hydrogen atom and n=4, the polymer is polycaprolactone. PHAs can include any number of monomers and commonly range from 100 to 30,000 monomers in length with molecular weight ranging from about 500 Daltons (Da) to over 1,000,000 Da.

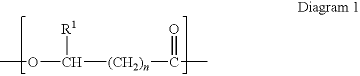

As with other polymers, PHA materials may be extruded into final product shape, dimension, and thickness. In one embodiment, the PHA material is extruded into a sheet having a diameter from about 0.01 millimeters to about 1.50 millimeters. Additionally, in one embodiment a PHA material is selected and extruded to include plurality of microscopic pores. In another embodiment, a first PHA material is provided with a first biodegradation rate, and a second PHA material is provided with a second biodegradation rate. In yet another embodiment, a third PHA material is provided with a third biodegradation rate.

Referring again to FIG. 1, following the first step 10 of collecting the excrement sample on the upper surface 102 of the decomposition container 100, the next step 12 includes treating the excrement with a bioactive agent. Generally, the bioactive agent is any culture or mixed culture of microorganisms capable of digesting the various components of the excrement. For example, the bioactive agent may include one or more microorganisms or components to include enzymes such as lipases, proteases and amylases, capable of digesting, or breaking-down the excrement into its basic chemical components. In one embodiment, the bioactive agent includes a microorganism that can digest the protein components of the excrement into small peptides and individual amino acids. In another embodiment, the bioactive agent includes a microorganism that can digest the lipid or fat components of the excrement into glycerol and fatty acids. In another embodiment, the bioactive agent includes a microorganism that can digest the carbohydrate components of the excrement into small organic molecules and individual elements of carbon, hydrogen, and oxygen. In another embodiment, the bioactive agent includes a microorganism that can digest the cellulose components of the excrement into small organic molecules and individual elements of carbon, hydrogen, and oxygen.

In yet another embodiment, the bioactive agent is a mixed culture including several microorganisms. For example, the bioactive agent may include bacterial microorganisms with extracellular enzymes. These enzymes, as well as free enzymes, include cellulase, protease, lipase, and amylase.

Referring again to FIG. 2B, the upper surface 102 of the top layer 104 may include a bioactive agent. For example, in one embodiment, the upper surface 102 of the top layer 104 is pretreated or pre-coated with the bioactive agent 120. In this embodiment, the bioactive agent 120 is either pre-applied to the upper surface 102 during manufacturing of the first material, or is applied during the assembly, or packaging of the decomposition container. In another embodiment, the bioactive agent 120 is applied to the upper surface 102 of the top layer 104 subsequent to manufacturing and packaging of the decomposition container 100. In this embodiment, the bioactive agent 120 may be applied either prior to the step 10 of collecting the excrement, or following the step 10 of collecting the excrement.

The bioactive agent 120 may be applied in a liquid form, a powder form, or a combination thereof. For example, a liquid preparation of the bioactive agent 120 may be prepared and stored in a spray bottle whereby a user may apply the bioactive agent 120 via the spray bottle. Alternatively, a powder preparation of the bioactive agent 120 may be prepared and stored in a container. The container may be configured to include a plurality of holes at one end such that by inverting the container and shaking and/or squeezing the container, the powder preparation may be applied to the decomposition container. In either embodiment, the bioactive agent 120 is deposited such that the bioactive agent 120 contacts the excrement.

Referring now to FIGS. 1 and 2B, the third step 14 is to provide oxygen to the bioactive agent 120. Decomposition of the excrement is accomplished by providing O₂ to the aerobic bioactive agent 120 to convert excrement to CO₂ and water, as well as provide new cell mass. In environmental chemistry, the Biochemical oxygen demand (BOD) is a measure of the amount of oxygen that bacteria will consume while decomposing organic matter under aerobic conditions. Biochemical oxygen demand may be determined by incubating the bioactive agent 120 and a portion of an excrement sample, in a sealed sample of water for five days and measuring the loss of oxygen from the beginning to the end of the test. The aerobic bioactive agent 120 feeds on the excrement sample within the sample of water while metabolizing the dissolved oxygen in the water sample. Dilutions of the bioactive agent 120 must be made prior to running the test to ensure that the bioactive agent 120 does not deplete the available oxygen before the end of the test. Results of the test are reported in milligrams of oxygen.

In one embodiment, a BOD of approximately 300 mg is required to decompose an average human excrement sample. Therefore, the bioactive agent 120 must be supplied with at least 300 mg of oxygen per excrement sample. Where the excrement and bioactive agent 120 are exposed to ambient air 124, the water from the excrement, the oxygen dissolved within the excrement, and the oxygen from the ambient air 124 may be sufficient to allow the bioactive agent 120 to decompose the excrement sample. However, optimal decomposition is obtained by providing additional O2 to the system, for example, by providing O2 from an oxidizing agent. Therefore, when the drawstring 108 is actuated, the excrement and bioactive agent 120 are enclosed within the decomposition container 100, and the bioactive agent 120 may become starved for oxygen. As such, the bioactive agent 120 may become inactive and unable to digest the excrement. Therefore, it may be necessary to supplement the oxygen supply of the enclosed decomposition container 100 by using chemical oxidants.

In one embodiment, a chemical oxidant 130 is provided within a lumen 132 of the decomposition container 100. The lumen 132 is defined as the space between top and bottom layers 104 and 114, wherein the lumen 132 is enclosed by one or more sealed junctions 134 between the top and bottom layers 104 and 114 of the decomposition container 100. The chemical oxidant 130 may include any chemical or combination of chemicals that produces oxygen when exposed to water, air, and/or a catalyst.

For example, in one embodiment the chemical oxidant 130 comprises sodium percarbonate. When exposed to water, the sodium percarbonate dissociates to form sodium carbonate and hydrogen peroxide. Hydrogen peroxide dissociates to water and O2 in the presence of a catalyst, for example a catalyst being potassium iodide (KI) or catalase. The produced oxygen is then released from this reaction into the lumen 132. As sodium percarbonate is the salt of a strong base and a weak acid, aqueous solutions of sodium percarbonate are quite alkaline with pH greater than 11. As such, dissociated sodium percarbonate provides alkaline hydrogen peroxide solutions which are known as strong oxidizing agents. Therefore, it is imperative that first material 150 provide separation between the oxidizing agents and the bioactive agent 120. For example, if the oxidizing agent contacts the bioactive agent, the bioactive agent will be killed. One of skill in the art will appreciate that other chemical or combinations of chemicals may be effectively used within the lumen 132 to accomplish the purposes of this invention.

The excrement and bioactive agent 120 are deposited on the upper surface 102 of the top layer 104 and, as such, occupy a first compartment 160 of the decomposition chamber 100. The first compartment 160 is separated from the lumen 132, or second compartment 162 of the decomposition chamber 100 by the top layer 104. The top layer 104 is comprised of a first material 150. The first material 150 is selected so as to accommodate a relationship between the bioactive agent 120 and the chemical oxidant 130.

For example, in one embodiment a first material 150 is selected to permit water from the first compartment 160 to pass through the first material 150 to the second compartment 162. As such, the water from the first compartment 160 may activate the chemical oxidant 130 of the second compartment 162. In this same embodiment, the first material 150 is further configured to permit oxygen, generated by the activated chemical oxidant 130, to pass through the first material 150 into the first compartment 160. As such, the oxygen from the second compartment 162 is made available to the bioactive agent 120 of the first compartment 160. The first material 150 is further selected to comprise plurality of one-way pores thus permitting the passage of a fluid from the first compartment 160 to the second compartment 162. Furthermore, the one-way pores prevent a disinfectant product of the second compartment 162 from passing into the first compartment 160 to disinfect the bioactive agent 120.

In another embodiment, a first material 150 is configured to include one or more one-way valves 170. The one-way valve 170 is provided to allow oxygen from the activated chemical oxidant 130 to pass through the first material 150 into the first compartment 160. The one-way valve 170 is configured to provide oxygen exchange from the second compartment 162 to the first compartment 160 while preventing a disinfectant product of the chemical oxidant 130 from passing into the first compartment 160. The first material 150 may further comprise plurality of one-way pores to permit passage of water from the first compartment 160 to the second compartment 162, as previously discussed. As such, water is made available to activate the chemical oxidant 130 of the second compartment 162. In another embodiment, a breakable vial 180 containing water 182 and one or more catalysts 184 is enclosed within the second compartment 162. The breakable vial 180 is crushed to release the water 182 and catalyst 184 into the chemical oxidant 130. As such, the released water 182 activates the chemical oxidant 130 to produce oxygen. Additionally, the one-way valve 170 may be used to provide water to the chemical oxidant 130. In this embodiment, the breakable vial 180 may include only a catalyst 184. Alternatively, the vial 180 may be omitted and the catalyst 184 added to the chemical oxidant 130. As such, the chemical oxidant 130 and the catalyst 184 are activated by the water as provided by the one-way valve 170.

The first material 150 is further selected to be biodegradable. The biodegradation rate of the first material 150 is selected based on the ability of the bioactive agent 120 to decompose the excrement sample. For example, in one embodiment the first material 150 is selected to biodegrade subsequent to the bioactive agent's 120 complete digestion of the excrement sample. In another embodiment, the first material 150 biodegrades 15 days after being exposed to the excrement and the bioactive agent 120. In another embodiment, the first material 150 biodegrades 3 to 4 days after being exposed to the excrement and the bioactive agent 120.

The first material 150 is further selected to biodegrade prior to the biodegradation of the second material 152. In one embodiment, the second material 152 is selected to biodegrade 3 months following the biodegradation of the first material 150. In another embodiment, the second material 152 is selected to biodegrade 1 week following the biodegradation of the first material 150.

Referring again to FIGS. 1 and 2B, the fourth step 16 is to disinfect the bioactive agent and pathogens of the system with a disinfecting agent. One of ordinary skill in the art will appreciate that the bioactive agent and the pathogens of the present system are collectively classified as microorganisms, however it is understood that pathogens also include viruses. One of ordinary skill in the art will further appreciate that additional pathogens and bacteria may be introduced into the present system independent of the disclosed components or steps of the present invention. For example, additional bacteria and pathogens may be introduced into the present system by an insect or an animal coming in contact with the system. Therefore, it is anticipated that the disinfecting agent of the present system will disinfect all pathogens present within the system.

Oxidizing agents are commonly used as disinfecting agents to kill or disinfect microorganisms. Oxidizing agents, such as chlorine, act by oxidizing the cell membrane of microorganisms, which results in a loss of structure and leads to cell lysis and death. A large number of disinfectants operate in this way. Hydrogen peroxide is commonly used as a disinfectant, or disinfecting agent. When hydrogen peroxide comes into contact with the catalase enzyme of a microorganism, the peroxide compound is broken down into a water molecule and a hydroxyl free radical molecule. The hydroxyl free radical thereafter oxidizes the membrane of the microorganism resulting in cellular death. Therefore, in one embodiment of the present invention, an oxidizing agent is provided that causes cellular death upon contact of the oxidizing agent with a microorganism of the system. In another embodiment, a chemical reaction of the oxidizing agent and water provides a disinfecting agent that causes cellular death upon contact of the disinfecting agent with a microorganism of the system. Alternatively, exothermic heat created by the bioactivity of the bioactive agent may also provide disinfecting temperatures, thereby eliminating the need to destroy the pathogens by another means.

As previously discussed, a first material 150 is selected to comprise a first biodegradation rate. Upon biodegradation of the first material 150, the contents of the first and second compartments 160 and 162 are combined. As such, the excrement byproducts, the pathogens, and the bioactive agent become exposed to the chemical oxidant 130 and/or disinfecting byproducts of the chemical oxidant 130. At this point, the disinfecting agent of the chemical oxidant 130 disrupt the cellular walls of the pathogens and bioactive agent 120 resulting in complete disinfection of all pathogens present within the system.

As previously discussed, the second material 152 is selected to comprise a rate of biodegradation that is slower than the biodegradation rate of the first material 150. Additionally, the biodegradation rate of the second material 152 is selected to be slower than the time needed for the disinfecting agent to disinfect the microorganisms of the system. For example, in one embodiment the process of decomposition requires 3 to 4 days, and the process of disinfecting the microorganisms of the system requires less than 1 day. Therefore, in this embodiment, the second material is selected to biodegrade no less than approximately 5 days following collection of the excrement sample. In another embodiment, the second material 152 is selected to biodegrade no less than approximately 1 day after the disinfection of the microorganisms of the system. In yet another embodiment, the second material 152 is selected to biodegrade at a period of time subsequent to the disinfection of the microorganisms of the system.

Referring again to FIG. 1, the last step 18 of the method is to dispose of the byproduct materials of the excrement sample, the bioactive agent, the pathogens, and the chemical oxidant 130. At this step 18 in the process, the excrement sample has previously been decomposed by the bioactive agent and the pathogens of the system. As such, the processed excrement sample generally comprises only small and simple chemical components or breakdown products of the original excrement. Additionally, the oxidizing agent 130 of the second compartment 162, and the contents of the first compartment 160 have been commingled resulting in the disinfection of the microorganisms within the system. Therefore, the byproduct of the system, termed humus, comprises decomposed organic material containing dead organisms and high molecular carbohydrates that are unable to be decomposed by enzymes.

Having been disinfected of harmful pathogens, the humus of the system may be disposed in any manner useful to the user. For example, the rich organic content of the humus may be useful as mulch, compost, or fertilizer. Additionally, the humus may be used as a landfill material. For example, in one embodiment the humus and the second material 152 are together buried underground. As such, the humus and the second material 152 further decompose and assimilate into the surrounding environment. In another embodiment, the humus and the second material 152 are used to fertilize a crop. In another embodiment, the humus and the second material 152 are deposited into a landfill.

Referring now to FIG. 2C, a representative cross-sectional end view of a decomposition container 200 is illustrated. In this embodiment 200, a third material 154 is selected and interposed between the first material 150 and the second material 152. The third material generally comprises a polymer material similar to that of the first and second material 150 and 152. The third material 154 is further selected to comprise a biodegradation rate that is faster than the biodegradation rate of the second material 152, and slower than the biodegradation rate of the first material 150. For example, in one embodiment the third material 154 biodegrades subsequent to the biodegradation of the first material 150, and prior to the biodegradation of the second material 152.

The third material 154 is positioned between the top layer 104 and the bottom layer 114, thereby forming a middle layer 144 of the decomposition container 200. The space between the top layer 104 and the middle layer 144 provides a third compartment 164 of the container 200, wherein the third compartment 164 is enclosed by one or more sealed junctions 134 between the middle layer 144, the top layer 104, and the bottom layer 114. In one embodiment, a second bioactive agent 190 is provided within the third compartment 164 of the decomposition container 200. The second bioactive agent 190 may include any microorganism capable of digesting unmetabolized products of the decomposed excrement sample. In one embodiment, the metabolic activity of second bioactive agent 190 is greater than the metabolic activity of a pathogen within the system. Alternatively, the metabolic activity of the second bioactive agent 190 may be equal to, or less than the metabolic activity of the pathogen. For example, in another embodiment, the metabolic activity of the second bioactive agent 190 is less than the metabolic activity of the pathogen, and thus the second bioactive agent 190 is provided in large quantities. As such, the large quantity of the second bioactive agent 190 comprises a cumulative metabolic activity greater than the metabolic activity of the pathogen.

The second bioactive agent 190 prevents a pathogen from metabolizing, and therefore surviving on any unmetabolized components of the excrement sample. For example, in one embodiment a bioactive agent 120 is provided to digest an excrement sample. In this embodiment, the bioactive agent 120 is unable to fully digest one or more component of the excrement sample and therefore leaves a portion of the excrement sample. As such, a pathogen of the excrement sample is able to survive by digesting the unmetabolized portion of the excrement sample. In this embodiment, the second bioactive agent 190 is provided to metabolize the remaining excrement sample, thereby depriving the pathogen of the food source. Therefore, following the biodegradation of the first material 150, the residual excrement sample, the pathogens, and the bioactive agent 120 are combined into the third compartment 164. Once combined, the second bioactive agent 190 begins metabolizing the unmetabolized food source of the pathogens and the first bioactive agent 120. As such, the excrement sample becomes completely digested and decomposed thereby depriving the microorganisms of any energy source within the decomposition container 200.

The third material 154 of the decomposition container 200 is selected to biodegrade subsequent to the biodegradation of the first material 150 and prior to the biodegradation of the second material 152. The first and third materials 150 and 154 may further comprise plurality of pores to permit passage of water through the first and third materials 150 and 154. For example, in one embodiment water from the excrement sample of the first compartment 160 passes through pores of the first material 150 and into the third compartment 164. In another embodiment, water from the third compartment 164 passes through pores of the third material 154 and into the second compartment 162. Alternatively, the third material 154 is impermeable to water and, therefore, the second compartment 162 comprises a breakable vial 180 having water 182. In another embodiment, the first and third materials 150 and 154 further comprise one or more one-way valves 170. As such, oxygen from the second compartment 162 may pass through the one-way valve 170 into the third compartment 164. Additionally, oxygen from the third compartment 164 may pass though the one-way valve 170 into the first compartment 160.

Upon biodegradation of the third material 154, the bioactive agent 120, the excrement sample, the second bioactive agent 190, and pathogens of the third compartment are combined with a chemical oxidant 130 within the second compartment 162. As previously discussed, oxygen radicals of the chemical oxidant 130 oxidize the cellular membranes of the microorganisms resulting in complete disinfection of all pathogens within the decomposition container 200.

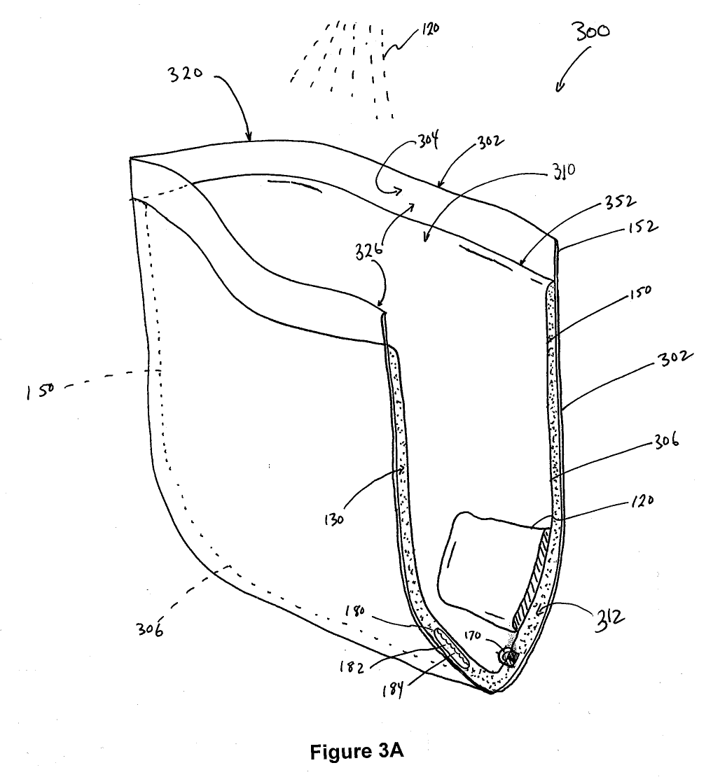

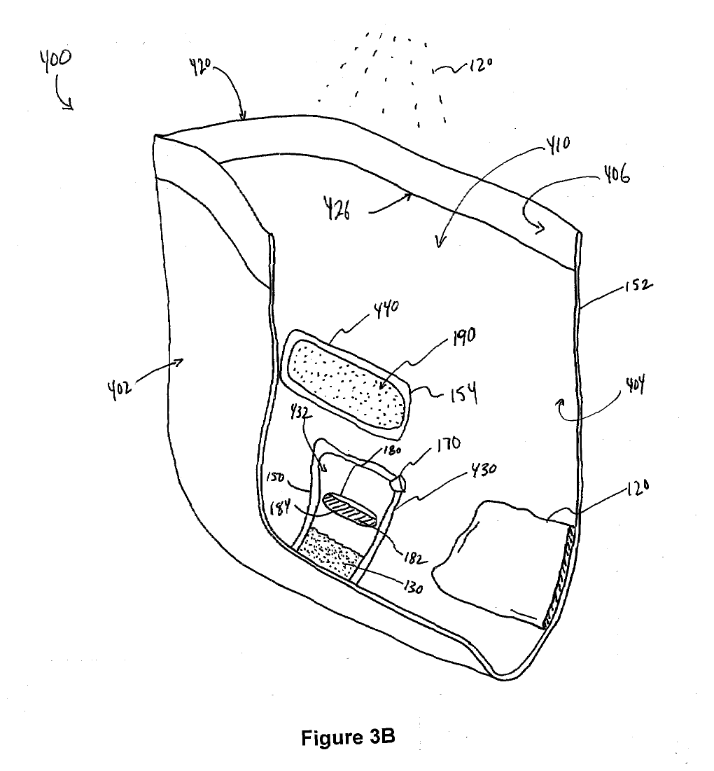

Referring now to FIG. 3A, a representative partially cross-sectioned perspective view of a decomposition container 300 is illustrated. The decomposition container 300 generally resembles a bag, wherein the container 300 comprises an outer covering 302, an inner compartment 310, and an opening 320. The container 300 further includes an inner surface 306 comprising a first material 150. The first material 150 includes properties and characteristics similar to those of the first material 150 discussed in connection with decomposition containers 100 and 200, above. The first material 150 is positioned within the outer covering 302 so as to line the interior of the decomposition container 300. The upper edge 352 of the first material 150 is sealed against an inner surface 304 of the outer covering 302. As such, a lumen or second compartment 312 is provided between the first material 150 and the outer covering 302. The outer covering 302 comprises a second material 152 having properties and characteristics similar to those of the second material 152 discussed in connection with decomposition containers 100 and 200, above.

The second compartment 312 is sealed at the upper edge 352. As such, the contents of the inner compartment 310 are separated from the contents of the second compartment 312. Therefore, in one embodiment the inner compartment 310 contains a bioactive agent 120 and the second compartment 312 contains a chemical oxidant 130. The characteristics of the bioactive agent 120 and the chemical oxidant 130 are similar to those of the bioactive agent 120 and the chemical oxidant 130 discussed in connection with decomposition containers 100 and 200, above. The bioactive agent 120 may either be pre-applied to the inner surface 306 of the container 300, or may be applied through the opening 320 following collection of an excrement sample.

The decomposition container 300 is utilized to collect an excrement sample. The excrement sample is inserted into the container 300 through the opening 320. The volume and geometry of the decomposition container 300 may be modified to accommodate any use of the bag. For example, in one embodiment the volume and geometry of the container 300 is configured to accommodate a single excrement sample. In another embodiment, the container 300 is configured to accommodate multiple excrement samples. In another embodiment, the geometry of the container 300 is configured to include a flat bottom such that the container 300 may support itself in an upright and opened position.

Once the excrement sample has been collected, the excrement is then treated with a bioactive agent 120. As previously discussed, the bioactive agent 120 may be pre-deposited, or pre-applied to the inner surface 306 of the container 300. As such, the excrement sample is disposed on top of the bioactive agent 120. Alternatively, the excrement sample is first collected and then a bioactive agent 120 is applied through the opening 320 of the container 300 to the outer surface of the excrement. In either embodiment, the bioactive agent 120 is made available to interact with the excrement sample.

Once the desired volume of excrement sample is collected, the container 300 is closed and sealed. In one embodiment, the inner surface 304 of the outer covering 302 is configured to include a closure device 326. In one embodiment the closure device 326 is an adhesive strip. In another embodiment the closure device 326 is a mating channel closure. In another embodiment the closure device 326 is a drawstring. In another embodiment, the closure device 326 comprises two or more drawstrings to facilitate making a dam for collecting the excrement from a flat sheet. As such, the closure device 326 eliminates the need for rigid support over that of the single drawstring approach.

The process of decomposition is similar to the decomposition process discussed in connection with FIGS. 1-2C, above. The bioactive agent 120 and the excrement sample may either be conjoined or admixed. The bioactive agent 120 is conjoined with the excrement sample by bringing the two components into contact with one another. Alternatively, the bioactive agent 120 and the excrement sample are admixed by physically manipulating the components to form a relatively homogenous mixture. The admixing may be accomplished either by directly contacting the components of the mixture with a paddle or a stick, or by indirectly contacting the components via the outer surface of the decomposition container 300. However, optimal admixing may be accomplished via a tool, such as a paddle.

Upon biodegradation of the first material 150, the contents of the inner compartment 310 combine with the chemical oxidant 130 of the second compartment 312. As such, free radicals of the chemical oxidant 130 disinfect the microorganisms of the system, as previously discussed above.

Referring now to FIG. 3B, a representative partially cross-sectioned perspective view of a decomposition container 400 is shown. The decomposition container 400 generally resembles a bag, wherein the container 400 comprises an outer surface 402, an inner surface 404, an inner compartment 410, and an opening 420. The container 400 is comprised of a second material 152 having properties similar to the second material 152 previously discussed in detail. The container 400 further comprises a first packet 430. The first packet 430 generally comprises a sealed container having a lumen or first compartment 432, the first packet 430 being comprised of a first material 150. The first material 150 includes properties and characteristics similar to the first material 150 previously discussed in detail.

The first packet 430 contains a chemical oxidant 130. The chemical oxidant 130 is provided to supply oxygen to a bioactive agent 120 within the container 400. The characteristics and properties of the bioactive agent 120 and the chemical oxidant 130 are similar to those of the agent 120 and oxidant 130 discusses in connection with the decomposition containers 100, 200, and 300, above. The first packet 430 may be further modified to include a one-way valve 170. The one-way valve 170 is provided to release oxygen from the first compartment 432 of the packet 430. Additionally, a breakable vial 180 may be included in the first compartment 432 to provide water 182 to the chemical oxidant 130, as previously discussed.

The decomposition container 400 is utilized to collect an excrement sample. The excrement sample is inserted into the container 400 through the opening 420. The volume and geometry of the decomposition container 400 may be modified to accommodate any use of the container 400. For example, in one embodiment the volume and geometry of the container 400 is configured to accommodate a single excrement sample. In another embodiment, the container 400 is configured to accommodate multiple excrement samples. In another embodiment, the geometry of the container 400 is configured to include a flat bottom such that the container 400 may support itself in an upright and opened position.

Once the excrement sample has been collected, the excrement is then treated with a bioactive agent 120. The bioactive agent 120 may either be pre-deposited, or pre-applied to the inner surface 404 of the container 400, or may be applied directly to the outer surface of the collected excrement sample. In either embodiment, the bioactive agent 120 is made available to interact with the excrement sample.

Once the desired volume of excrement is collected, the container 400 is closed and sealed. In one embodiment, an upper edge 406 of the inner surface 404 is configured to include a closure device 426. In one embodiment, the closure device 426 is an adhesive strip. In another embodiment the closure device 426 is a mating channel closure. In another embodiment the closure device 426 is a drawstring.

The process of decomposition is similar to the decomposition process discussed in connection with the disclosure above. The bioactive agent 120 and the excrement sample may either be conjoined or admixed, as described above. Following the complete decomposition of the excrement sample, the first material 150 of the first packet 430 biodegrades and the contents of the first packet 430 combine with the contents of the inner compartment 410. The microorganisms of the inner compartment 410 are then disinfected by the free radicals of the chemical oxidant 130, as previously discussed.

In another embodiment, a second packet 440 is added to the inner compartment 410 of the decomposition container 400. In this embodiment, the second packet 440 contains a second bioactive agent 190. The second bioactive agent 190 is provided to ensure that any unmetabolized excrement is metabolized, thereby depriving any pathogen of nutrients within the system. The characteristics and properties of the second bioactive agent 190 are similar to those of the second bioactive agent 190 previously discussed.

The second packet 440 comprises a third material 154. The third material 154 is selected to biodegrade prior to the biodegradation of the first material 150 of the first packet 430. Furthermore, the first material is selected to biodegrade subsequent to the biodegradation of the third material 154, and prior to the biodegradation of the second material 152. Specifics regarding the material properties and characteristics may be found in connection with the previous discussion regarding the first, second, and third material 150, 152, and 154, above.

Therefore, following decomposition of the excrement sample by the bioactive agent 120, the third material 154 of the second packet 440 biodegrades and the second bioactive agent 190 is combined with the contents of the inner compartment 410. Once the second bioactive agent 190 metabolizes the unmetabolized components of the excrement sample, the first material 150 of the first packet 430 biodegrades and the contents of the first packet 430 are combined with the contents of the inner compartment 410. As such, the free radicals of the chemical oxidant 130 disinfect the microorganisms of the container 400, in accordance with the previous discussion.

One of skill in the art will appreciate that any embodiment of the present invention may include any of the presently disclosed features, functions, or elements and remain within the scope of the invention. Additionally, one of skill in the art will appreciate that the bioactive agent of the present invention may be expanded to include an enzyme, or other non-microorganistic entities to aid in decomposing the excrement sample.

Furthermore, the bioactive agent of the present invention may be embodied in any variety of forms. For example, in one embodiment, the bioactive agent is lyophilized, or otherwise preserved to prevent premature metabolic, or biological activity. In another embodiment, the bioactive agent is vacuum sealed to protect the agent against exposure to moisture and oxygen in the ambient air.

In another embodiment, the bioactive agent is combined with a filler and/or an excipient to form a pill or cake. In this embodiment, the bioactive agent is then flushed or otherwise introduced into a sewer system to decompose excrement therein. In another embodiment, the bioactive agent is introduced into a septic system. In another embodiment, the bioactive agent is introduced to excrement during the chemical treatment of the excrement by a water treatment plant. Alternatively, a filler may be used to introduce the bioactive agent into the decomposition container, as discussed above.

In another embodiment, an envisioned product starts with an O₂ oxidizer generator positioned on the bottom of the product. The next layer up includes a bioactive agent. Excrement is then collected on top of the bioactive agent. Additionally, another bag of oxidizer is provided on top of the excrement; however this oxidizer has no contact with any catalyst of the system. Rather, the top or second oxidizer produces hydrogen peroxide that is used to disinfect the excrement. This is accomplished due to weight of hydrogen peroxide being heavier than air; therefore the hydrogen peroxide will not leak off into the air. Furthermore, each of the aforementioned components may be contained in materials having varying rates of biodegradation so as to regulate the exposure of each component to other components of the system or product.

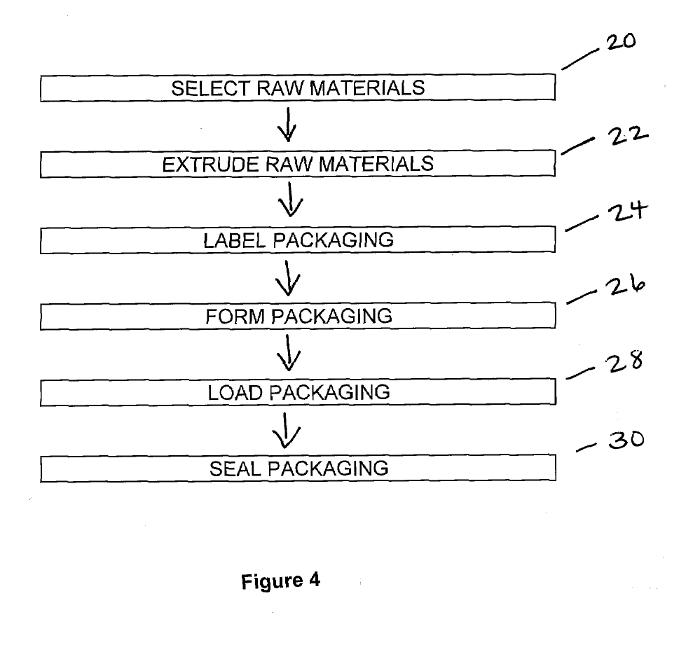

Referring now to FIG. 4, a representative flow chart is shown for manufacturing the decomposition container of the present invention. The process of manufacturing the container may be accomplished as individual steps, or may be automated into a continuous process. Prior to manufacturing the container, the first step 20 is to select the raw material of the decomposition container. As previously discussed, the material of the decomposition container includes a biodegradable biopolymer comprising hydroxyalkonate monomers. The hydroxyalkonate monomers may be selected and combined in any order necessary to accomplish the needs of the material. For example, in one embodiment a single monomer is selected and synthesized to provide a homogenous biopolymer material. In another embodiment, two or more monomers are selected and synthesized to provided a heterogeneous biopolymer material.

The polymer material may be synthesized by any method or technique known in the art of polymer science.

The first step 22 to manufacture the container is to extrude the synthesized material into sheets or tubes for the container. Extrusion is the process of compacting and melting the selected biopolymer material and forcing it through an orifice in a continuous fashion. In the present method, the orifice comprises a die, or other shaping device for molding the melted biopolymer material into a sheet material.

The extruded sheets are then trimmed or otherwise prepared to be processed into the final decomposition container. In one embodiment, the extruded sheet material is labeled or printed 24 prior to being assembled as a decomposition container. The label or printed information may include instructions, information regarding the source of the container, and artwork.

The step of assembling the decomposition container 26 largely depends upon the final embodiment of the container. For example, each component of the various embodiments require different offline operations. For example, where the oxidizing agent is supplied in a separate container, the oxidizer container is first formed on conventional converting machinery. The film is then oriented vertically and filled with the chemical oxidant. Following this step, the top of the container is sealed and the container is cut to the necessary length.

In another line the breakable vial or a biodegradable bag for containing the water and catalysts is made on a converting line. The vial or bag is then oriented vertically and filled with aqueous KI, catalase enzyme, and/or water. Following this step, the container is sealed and cut to length.

Still another converting line provides the bioactive agent, where the bioactive agent is contained within a lumen of the system. Again, these bags are created, oriented vertically and filled with the bioactive agent. The bags are then sealed and cut to the appropriate dimensions. Where the decomposition container is the container shown in FIG. 2A, there are several off line operations that need to be performed.

The material for each container is unrolled and fed to a printing station that provides all the labeling. From here the film goes to the next station that applies adhesive in the machine and cross machine directions. The next station lays down the oxidizing agent bag. Next, adhesive is placed on the upper part of this bag. Following this step the bioactive agent bag is positioned on top of the oxidizing agent bag. As the line moves forward, the bag sheet is cut to length and stacked up for further processing. Offline, the drawstring assembly is made on automatic equipment. The resultant product is a flexible tube of material containing a drawstring.

The bag assemblies are loaded into an automated machine that places a rigid, yet flexible channel around the sheet. Next, the drawstring assembly is positioned on the sheet. Then the hem is formed to seal the sheet. The product is then released from the machine and packaged for shipment.

The final product provides an apparatus with a bottom portion that is impervious, and a top layer that is microporous for vapor transmission only. The bottom of the bioactive agent bag is microporous such that oxygen from the chemical oxidant bag can be accessed by the bioactive agent. However, one of skill in the art will appreciate that various methods may alternative designs may be incorporated to accomplish the purpose of the present invention.

Thus, as discussed herein, embodiments of the present invention relate to waste management and waste disinfection. More particularly, at least some embodiments of the present invention pertains to systems and methods for disinfecting excrement, including systems and methods for recycling excrement into a usable product.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A method for disinfecting an excrement sample, the method comprising:
decomposing the excrement sample with a bioactive agent;
providing oxygen to the bioactive agent; and
disinfecting one or more pathogens of the excrement sample with a disinfecting agent.

2. The method of claim 1, further comprising the step of disinfecting the bioactive agent with the disinfecting agent.

3. The method of claim 1, wherein the bioactive agent digests the organic chemical components of the excrement sample.

4. The method of claim 1, wherein the excrement sample is collected in a decomposition container.

5. The method of claim 4, wherein the bioactive agent is applied to at least one outer surface of the excrement sample.

6. The method of claim 5, wherein the bioactive agent is admixed with the excrement sample.

7. The method of claim 4, wherein the excrement sample and the bioactive agent are contained in a first compartment of the decomposition container, and the disinfecting agent is contained in a second compartment of the decomposition container.

8. The method of claim 7, wherein the first compartment comprises a material through which liquid of the excrement sample can pass into the second compartment of the decomposition container.

9. The method of claim 8, wherein the second compartment comprises a material through which oxygen generated by the disinfecting agent can pass into the first compartment of the decomposition container.

10. The method of claim 7, wherein a material between the excrement sample and the disinfecting agent biodegrades prior to the biodegradation of an outer material of the decomposition container.

11. The method of claim 10, wherein following the biodegradation of the material between the excrement sample and the disinfecting agent, the disinfecting agent contacts a mixture of the bioactive agent and the one or more pathogens of the excrement sample and disinfects the mixture.

12. The method of claim 1, wherein the bioactive agent comprises at least one of a microorganism and an enzyme.

13. The method of claim 1, wherein the disinfecting agent is sodium percarbonate.

14. The method of claim 1, wherein the byproduct material of the method is used as a fertilizer.

15. A device for disinfecting an excrement sample, the device comprising:
a first compartment for containing the excrement sample and a bioactive agent;
a second compartment for containing a disinfecting agent;
a first material interposed between the first compartment and the second compartment;
an outer surface of the device comprising a second material, the outer surface being used to contain at least a portion of the first compartment and the second compartment; and
means for securing the excrement sample, the bioactive agent, and the disinfecting agent within the device, wherein the first material biodegrades prior to the biodegradation of the second material such that the biodegradation of the first material causes the disinfecting agent to contact the excrement sample and the bioactive agent, and to disinfect the bioactive agent and one or more pathogens of the excrement sample.

16. The device of claim 15, wherein the first material further comprises a plurality of microscopic pores such that a liquid of the excrement sample can pass into the second compartment of the device.

17. The device of claim 16, wherein the first material further comprises a plurality of microscopic pores such that oxygen from the second compartment can pass into the first compartment of the device.

18. The device of claim 15, wherein the second compartment further comprises a defeatable valve for releasing oxygen from the second compartment.

19. The device of claim 15, wherein the second compartment further comprises a defeatable valve for providing water to the oxidizing agent.

20. The device of claim 18, wherein the defeatable valve is defeated by a threshold pressure within the second compartment of the device.

21. The device of claim 15, wherein the second compartment further comprises a breakable vial containing at least one of water and a catalyst, such that when the vial is broken, the contents are released and activate the disinfecting agent of the second compartment.

22. The device of claim 21, wherein the second compartment further comprises a microporous membrane for releasing oxygen from the second compartment.

23. The device of claim 21, wherein the second compartment further comprises a defeatable valve for releasing oxygen from the second compartment.

24. The device of claim 23, wherein the defeatable valve is defeated by a threshold pressure within the second compartment of the device.

25. The device of claim 16, wherein the means for securing the excrement sample, the bioactive agent, and the disinfecting agent comprises at least one of a draw-string, an adhesive, and a mating channel closure.

26. The device of claim 16, wherein the bioactive agent comprises at least one of a microorganism and an enzyme.

27. The device of claim 16, wherein the disinfecting agent is sodium percarbonate.

28. The device of claim 16, wherein the byproduct material of the method is used as a fertilizer.

29. A device for disinfecting an excrement sample, the device comprising:
a first compartment for containing the excrement sample and a first bioactive agent;
a second compartment for containing a second bioactive agent;
a first material interposed between the first compartment and the second compartment;
an outer surface of the device comprising a second material, the outer surface being used to contain at least a portion of the first compartment and the second compartment; and
means for securing the excrement, the first bioactive agent, and the second bioactive agent within the device, wherein the first material biodegrades prior to the biodegradation of the second material such that the biodegradation of the first material causes the second bioactive agent to contact the excrement sample and the first bioactive agent, and to digest any residual excrement sample available to one or more pathogens of the excrement sample, thereby depriving the one or more pathogens of any residual excrement sample.

30. The device of claim 29 further comprising a third compartment containing a disinfecting agent, the third compartment comprising a third material that biodegrades prior to the biodegradation of the second material such that upon biodegradation of the third material the disinfecting agent is released and disinfects the first bioactive agent, the second bioactive, and the one or more pathogens of the excrement sample.

31. The device of claim 29, wherein the second bioactive agent comprises at least one microorganism that is more bioactive than the first bioactive agent and the one or more pathogens of the excrement sample.

32. The device of claim 30 wherein the third material further comprises a plurality of microscopic pores such that a liquid of the excrement sample can pass into the third compartment of the device.

33. The device of claim 30, wherein the third material further comprises a plurality of microscopic pores such that oxygen from the third compartment can pass into the first compartment of the device.

34. The device of claim 30, wherein the third compartment further comprises a valve for releasing oxygen from the third compartment.

35. The device of claim 30, wherein the third compartment further comprises a breakable vial containing at least one of water and a catalyst, such that when the vial is broken the contents are released and activate the disinfecting agent of the third compartment.

36. A method for manufacturing a device for disinfecting an excrement sample, the method comprising:
selecting a first material for a first compartment;
selecting a second material for a second compartment;
extruding the first material into a first sheet material;
extruding the second material into a second sheet material;
inserting a disinfecting agent into the second sheet material, and then sealing the second sheet material to form the second compartment;
forming the first compartment from the first sheet material, and then inserting the second compartment into the first compartment; and
providing means for securing the second compartment within the first compartment.

37. The method of claim 36, wherein the manufacturing of the device is continuous.

38. The method of claim 36, wherein the first material is selected to biodegrade prior to the biodegradation of the second material.

39. The method of claim 38, wherein the disinfecting agent is sodium percarbonate.

40. The method of claim 36, further comprising:
selecting a third material for a third compartment;
extruding a third material into a third sheet material;
inserting a second bioactive agent into the third sheet material, and then sealing the third sheet material to form the third compartment; and
inserting the third compartment into the first compartment;
wherein the third material is selected to biodegrade prior to the biodegradation of the first material such that the second bioactive agent is released prior to the release of the disinfecting agent, the second bioactive agent thereafter digesting any residual excrement sample available to one or more pathogens of the excrement sample, thereby depriving the one or more pathogens of any residual excrement sample.

41. A device for disinfecting an excrement sample, the device comprising:
a first compartment for containing the excrement sample, a bioactive agent, and a second compartment, the second compartment comprising a first material, and the first compartment comprising a second material;
a disinfecting agent deposited within the second compartment; and
means for securing the excrement sample, the bioactive agent, and the second compartment within the first compartment, wherein the first material biodegrades prior to the biodegradation of the second material such that the biodegradation of the first material causes the disinfecting agent to contact the excrement sample and the bioactive agent, and to disinfect the bioactive agent and one or more pathogens of the excrement sample.

42. A device for disinfecting an excrement sample, the device comprising:
a first compartment for containing the excrement sample, a first bioactive agent, and a second compartment, the second compartment comprising a first material, and the first compartment comprising a second material;
a second bioactive agent deposited within the second compartment; and
means for securing the excrement sample, the first bioactive agent, and the second compartment within the first compartment, wherein the first material biodegrades prior to the biodegradation of the second material such that the biodegradation of the first material causes the second bioactive agent to contact the excrement sample and the bioactive agent, and to digest any residual excrement sample available to one or more pathogens of the excrement sample, thereby depriving the one or more pathogens of any residual excrement sample.

43. A device for disinfecting an excrement sample, the device comprising:
a first compartment for containing the excrement sample and a first bioactive agent;
a second compartment for containing a second bioactive agent;
a third compartment for containing a disinfecting agent;
a first material interposed between the first compartment and the second compartment;
a second material interposed between the second compartment and the third compartment;
an outer surface of the device comprising a third material, the outer surface being used to contain at least a portion of the first compartment, the second compartment, and the third compartment; and
means for securing the excrement sample, the first bioactive agent, the second bioactive agent, and the disinfecting agent within the device, wherein the first material biodegrades prior to the biodegradation of the second material, and the second material biodegrades prior to the biodegradation of the third material such that the biodegradation of the first material causes the second bioactive agent to contact the excrement sample and the first bioactive agent, and to digest any residual excrement sample available to one or more pathogens of the excrement sample, thereby depriving the one or more pathogens of any residual excrement sample, and such that the biodegradation of the second material causes the disinfecting agent to contact the excrement sample, the first bioactive agent, the second bioactive agent, and the one or more pathogens of the excrement sample, and to disinfect the first bioactive agent, the second bioactive agent, and the one or more pathogens of the excrement sample.

44. A device for disinfecting an excrement sample, the device comprising:
a first compartment for containing the excrement sample, a first bioactive agent, a second compartment, and a third compartment, the second compartment comprising a first material, the third compartment comprising a second material, and the first compartment comprising a third material;
a second bioactive agent deposited within the second compartment;
a disinfecting agent deposited within the third compartment; and
means for securing the excrement sample, the first bioactive agent, the second compartment, and the third compartment within the first compartment, wherein the first material biodegrades prior to the biodegradation of the second material, and the second material biodegrades prior to the biodegradation of the third material such that the biodegradation of the first material causes the second bioactive agent to contact the excrement sample and the first bioactive agent, and to digest any residual excrement sample available to one or more pathogens of the excrement sample, thereby depriving the one or more pathogens of any residual excrement sample, and such that the biodegradation of the second material causes the disinfecting agent to contact the excrement sample, the first bioactive agent, the second bioactive agent, an the one or more pathogens of the excrement sample, and to disinfect the first bioactive agent, the second bioactive agent, and the one or more pathogens of the excrement sample.
